(19) United States
(12) Patent Application Publication
(10) Pub. No.: US 2025/0339052 A1
(43) Pub. Date: Nov. 6, 2025

(54) A TUBE FOR CONVEYING A FLUID

(71) Applicant: Apnea-Tech Limited, Merseyside (GB)

(72) Inventors: Benjamin Walsh, Lincolnshire (GB); Andrew Moss, Cheshire (GB)

(73) Assignee: Apnea-Tech Limited, Merseyside (GB)

(21) Appl. No.: 18/854,231

(22) PCT Filed: Mar. 31, 2023
(86) PCT No.: PCT/IB2023/053275

§ 371 Date: Oct. 4, 2024

(30) Foreign Application Priority Data
Apr. 6, 2022 (GB) 2205020.7

(51) Int. Cl. A61B 5/087; A61M 16/20

(52) U.S. Cl. CPC A61B 5/0876; A61M 16/208

(57) ABSTRACT

Aspects of the present invention relate to a tube for conveying a fluid. the tube comprising: a first passage: a second passage comprising a pressure sensor connection point through a wall of the second passage: and a valve configured to be in an open condition when a fluid flow through the tube in is a first direction and configured to be in a closed condition when the fluid flow through the tube is in a second direction, opposite to the first direction. wherein the pressure sensor connection point is upstream of the valve. when the fluid flow is in the first direction, and downstream of the valve. when the fluid flow is in the second direction: wherein. when the valve is in the open condition fluid can be conveyed via the first passage. and when the valve is in the closed condition fluid cannot be conveyed via the first passage.

### TECHNICAL FIELD

The present disclosure relates to a tube for conveying a fluid. Particularly, but not exclusively, the present disclosure relates to a tube for conveying a fluid in an obstructive sleep apnoea screening device, and an obstructive sleep apnoea screening device.

Aspects of the invention relate to a tube, and to a device.

### BACKGROUND

Sleep apnoea is a condition that causes the sufferer to temporarily stop breathing while asleep. Left untreated it can lead to a number of conditions, from chronic fatigue to cardiovascular disorders.

It is known to provide screening devices for checking breathing during sleep, such devices measuring airflow through a nasal cannula connected to a pressure sensor.

It is an aim of the present invention to address one or more of the disadvantages associated with the prior art.

### SUMMARY OF THE INVENTION

Aspects and embodiments of the invention provide a tube, and a device as claimed in the appended claims.

According to an aspect of the present invention there is provided a tube for conveying a fluid, the tube comprising: a first passage; a second passage comprising a pressure sensor connection point through a wall of the second passage; and a valve configured to be in an open condition when a fluid flow through the tube in is a first direction and configured to be in a closed condition when the fluid flow through the tube is in a second direction, opposite to the first direction, wherein the pressure sensor connection point is upstream of the valve, when the fluid flow is in the first direction, and downstream of the valve, when the fluid flow is in the second direction; wherein, when the valve is in the open condition fluid can be conveyed via the first passage, and when the valve is in the closed condition fluid cannot be conveyed via the first passage.

An advantage of this aspect of the invention is that the pressure detected when the fluid flows in the first direction is different from when the fluid flows in the second direction, as there is a greater fluid flow past the pressure sensor when the fluid flows in the second direction, and therefore the direction of fluid flow can be more easily determined.

When the valve is in the open condition fluid may be conveyed via the first passage and the second passage, and when the valve is in the closed condition fluid may only be conveyed via the second passage. This provides an advantage of altering the fluid flow acting in a second direction, where in the second direction the valve is closed, thereby directing the fluid to flow through the second passage only. The relative differences in pressure in a bi-directional flow can therefore be passively amplified by this aspect of the invention.

The tube may comprise a pressure sensor attached to the pressure sensor connection point for measurement of pressure of the fluid flow through the second passage of the tube.

The pressure sensor may have a pressure sensing element in the flow of fluid inside of the second passage.

The pressure sensor connection point may be a pressure sensor tap. The pressure sensor tap may be connected to a pressure sensing element outside of the second passage.

The tube may be circular in cross section.

The first passage may be annular in cross section and have a first outer diameter, the second passage may be circular in cross section and may have a second outer diameter. The second passage may be concentric with the first passage. The second outer diameter may be less than the first outer diameter. The valve may be annular in cross section, having a valve outer diameter in the closed condition that is substantially equal to the first outer diameter and a valve inner diameter in the closed condition that is substantially equal to the second outer diameter.

The first passage may be a first segment of the tube in cross section forming a first part of the tube, the second passage may be a second segment of the tube in cross section forming a second part of the tube, and the valve may be a segment in cross section when in the closed condition, encompassing substantially the same cross section as the first passage.

The first segment may be a semi-circle forming a first half of the tube in cross section and the second segment may be a semi-circle forming a second half of the tube in cross section.

The second passage may comprise an expansion chamber. This provides an advantage of further modifying the pressure difference between the fluid flow in the first direction and the fluid flow in the second direction.

The second passage may comprise a restrictor plate such that the pressure sensor connection point is between the restrictor plate and the valve.

The restrictor plate may comprise a drain hole for draining a liquid condensate from the tube

According to an aspect of the present invention there is provided a device comprising a tube according to any preceding aspect and a pressure sensor according to a preceding embodiment and further comprising a processor for receiving signals from the pressure sensor, a memory and power source.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1 illustrates a section of tube according to an embodiment of the invention, with an open valve;

FIG. 2 illustrates the section of tube of FIG. 1 with a closed valve;

FIG. 3 illustrates a cross section of the tube of Figure a and FIG. 2 at section X1-X2;

FIG. 4 illustrates a cross section of the tube of FIG. 2 at a valve portion of the tube at section Y1-Y2;

FIG. 5 illustrates a section of tube according to an embodiment of the invention, with an open valve;

FIG. 6 illustrates the section of tube of FIG. 5 with a closed valve;

FIG. 7 illustrates a cross section of the tube of FIG. 5 and FIG. 6 at section X3-X4;

FIG. 8 illustrates a cross section of the tube of FIG. 6 at a valve portion of the tube at section Y3-Y4;

FIG. 9 illustrates a section of tube according to an embodiment of the invention, with an open valve;

FIG. 10 illustrates a section of tube according to an embodiment of the invention, with an open valve;

FIG. 11 illustrates a section of tube according to an embodiment of the invention, with an open valve;

FIG. 12 illustrates a cross section of the tube of FIG. 11 at a restrictor plate portion of the tube at section X5-X6;

FIG. 13 illustrates a section of tube according to an embodiment of the invention, with an open valve;

FIG. 14 illustrates a cross section of the tube of FIG. 13 at section X7-X8; and

FIG. 15 shows a device in accordance with an embodiment of the invention.

In the drawings, like parts are denoted by like reference numerals.

### DETAILED DESCRIPTION

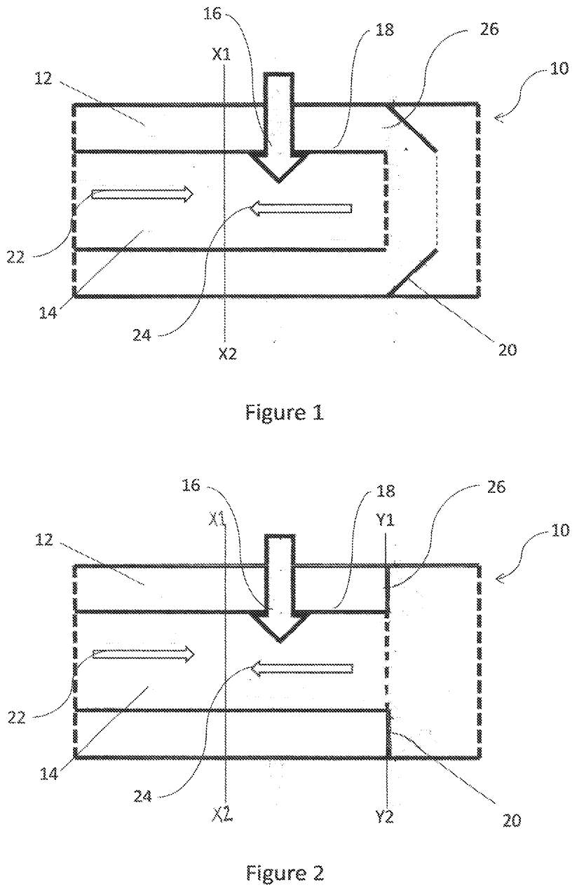

Examples of the present disclosure relate to a tube. In particular, examples of the present invention relate to a dual passage tube, though it should be understood that a different number of passages may be provided. Non-limiting examples will now be described with reference to accompanying FIGS. 1 to 15, where the figures illustrate a tube 10, 110, 210, 310, 410, 510 and a device 100.

The tube 10, 110, 210, 310, 410, 510 of the present invention is intended for use in an obstructive sleep apnoea device 100, where the obstructive sleep apnoea device 100 comprises the tube 10, 110, 210, 310, 410, 510, a memory 104, a power source 106, a processor 108 and either a sensor 102 positioned outside of the tube 10, 110, 210 or a sensor 316 positioned inside the tube 310, 410, 510. The obstructive sleep apnoea device 100 may provide the function of a screening device for measuring parameters relating to sleep apnoea. The device may amplify the difference in pressure of the flow between a forward and a reverse flow direction, without the requirement of active amplification.

With reference to FIG. 1, there is shown a tube 10 with two passages 12, 14 functioning as fluid channels, each passage 12, 14 being able to convey fluid, such as a gas or a liquid, between two points in a fluid transfer system.

Tube 10 comprises a first passage 12 and a second passage 14. The second passage 14 has a pressure sensor connection point 16 through a wall 18 of the second passage 14. The pressure sensor connection point 16 may be a pressure sensor tap. The pressure sensor tap may be connected to a pressure sensing element 102 outside of the second passage 14.

The tube 10 comprises a valve 20 configured to be in an open condition, as shown in FIG. 1, when a fluid flow through the tube 10 in is a first direction 22 and configured to be in a closed condition, as shown in FIG. 2, when the fluid flow through the tube 10 is in a second direction 24, opposite to the first direction 22. In FIG. 1 and FIG. 2 the first direction 22 is from left to right and the second direction 24 is from right to left.

The pressure sensor connection point 16 is upstream of the valve 20 when the fluid flow is in the first direction 22. The pressure sensor connection point 16 is downstream of the valve 20, when the fluid flow is in the second direction 24.

The valve 20 comprises a moveable part that is actuated between the open condition and the closed condition due to forces exerted upon the valve 20 by the flow of fluid. When fluid flows in the first direction 22, the valve 20 is forced to open, as shown in FIG. 1, under the action of the fluid, such that fluid flows through both the first passage 12 and the second passage 14. When fluid flows in the second direction 24, the valve 20 is forced to close, as shown in FIG. 2, under the action of the fluid such that fluid can only flow through the second passage 14 as the valve 20 closes the aperture 26 of the first passage 12 preventing fluid passage therethrough.

In examples of the invention, when the valve 20 is in the open condition fluid can be conveyed via the first passage 12, and when the valve 20 is in the closed condition fluid cannot be conveyed via the first passage 12. In various embodiments the tube may comprise a plurality of passages, one or more of which is prevented from conveying fluid when the valve 20 is in the closed condition, whilst one or more of the passages may allow the passage of fluid when the valve 20 is in the closed condition.

In the example of FIG. 1 and FIG. 2, when the valve 20 is in the open condition fluid can be conveyed via both the first passage 12 and the second passage 14, and when the valve 20 is in the closed condition fluid cannot be conveyed via the first passage 12, but can only be conveyed via the second passage 14. Given a fluid transfer system where the same volume of fluid must pass through the tube in the first direction 22 and the second direction 24, for example in a sleep apnoea screening device where a user breathes in and out through a mask connected to the tube 10, the closing of the valve 20 to prevent fluid passing through the first passage 12 when fluid flow through the tube 10 is in the second direction 24 means that all of the fluid passes through the second passage 14 in the second direction 24. In such a scenario, a different fluid pressure is present at the pressure sensor connection point 16 during fluid flow in the first direction 22 and fluid flow in the second direction 24, thus providing an indicator of the direction of flow of the fluid, making the direction of fluid flow easier to detect using only a single sensor.

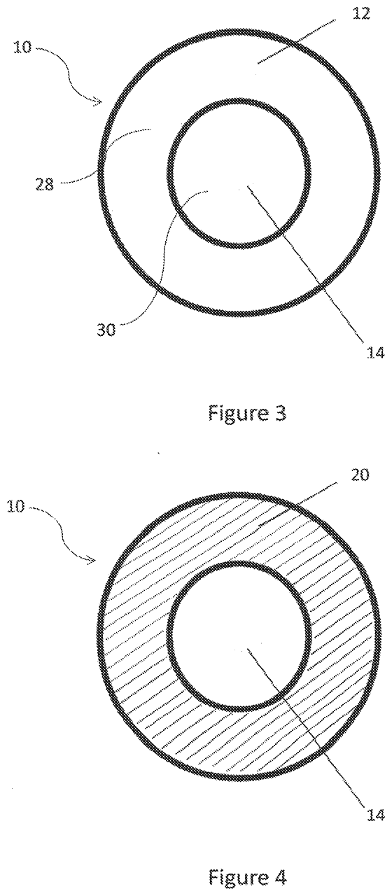

FIG. 3 shows a cross section of the tube 10 through section X1-X2 of FIG. 1. In this example the tube 10 is circular, or annular providing a circular inner channel, in cross section. In this example the first passage 12 is annular in cross section, that is the first passage 12 is an annular section 28 of the tube 10, and the second passage 14 is circular in cross section, that is the second passage 14 is a circular section 30 of the tube 10.

The first passage 12, which is annular has an outer diameter and an inner diameter. The second passage 14, which is circular, has an outer diameter, which is substantially congruent with the inner diameter of the first passage 12. In FIG. 3, the second passage 14 is concentric with the first passage 12, though this may not be the case in some embodiments.

The outer diameter of the first passage 12 is less than the outer diameter of the second passage 14. The cross section of the tube 10, along the length of the tube 10 where there is a first passage 12 and a second passage 14, may be consistent, that is, the outer diameter of the first passage 12 and the outer diameter of the second passage 14 may remain constant along such a length of tube 10.

FIG. 4 shows a cross section of the tube 10 through section Y1-Y2 of FIG. 2. In this example, where the valve 20 is in the closed position, the valve 20 is annular in cross section. The valve 20, which may be formed of a flexible material to move from the open condition shown in FIG. 1 to the closed condition in FIG. 2, has an outer diameter in the closed condition that is substantially equal to the first outer diameter of the first passage 12 and an inner diameter in the closed condition that is substantially equal to the second outer diameter, that is the outer diameter of the second passage 14. Thereby, when the valve 20 is in the closed condition the first passage 12 is entirely closed off from the fluid path and so fluid conveyed in the second direction 24 can only pass through the second passage 14.

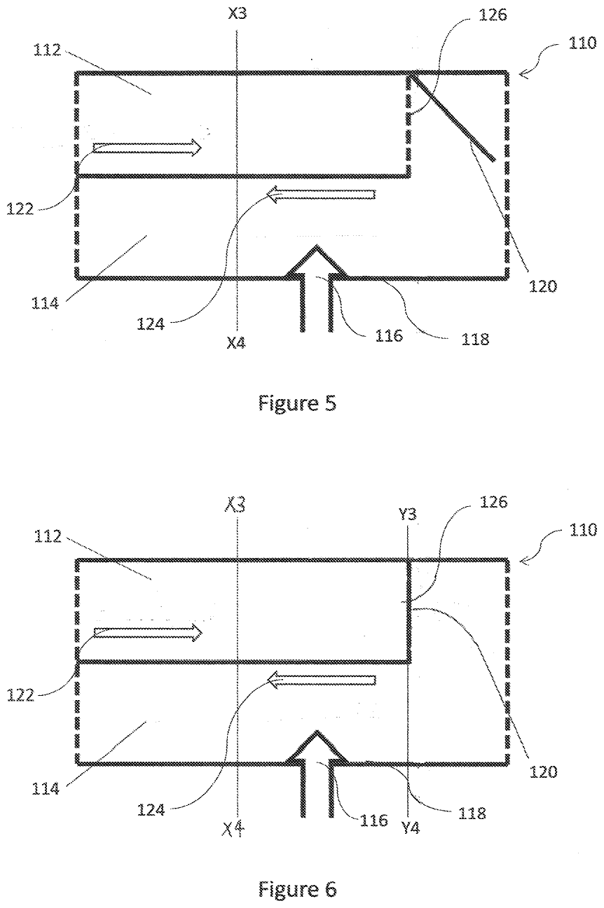

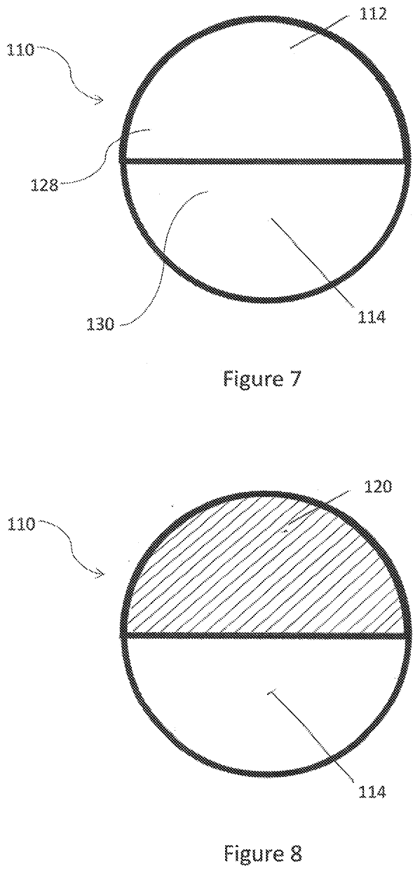

An alternative embodiment is shown in FIG. 5, FIG. 6, FIG. 7, and FIG. 8.

With reference to FIG. 5, there is shown a tube 110 with two passages 112, 114 functioning as fluid channels, each passage 112, 114 being able to convey fluid, such as a gas or liquid, between two points in a fluid transfer system.

The tube 110 comprises a first passage 112 and a second passage 114. The second passage 114 has a pressure sensor connection point 116 through a wall 118 of the second passage 114.

The tube 110 comprises a valve 120 configured to be in an open condition, as shown in FIG. 5, when a fluid flow through the tube 110 in is a first direction 122 and configured to be in a closed condition, as shown in FIG. 6, when the fluid flow through the tube 110 is in a second direction 124, opposite to the first direction 122. In FIG. 5 and FIG. 6 the first direction 122 is from left to right and the second direction 124 is from right to left.

The pressure sensor connection point 116 is upstream of the valve 120 when the fluid flow is in the first direction 122. The pressure sensor connection point 116 is downstream of the valve 120, when the fluid flow is in the second direction 124.

The valve 120 comprises a moveable part that is actuated between the open condition and the closed condition due to forces exerted upon the valve 120 by the flow of fluid. When fluid flows in the first direction 122, the valve 120 is forced to open, as shown in FIG. 5, under the action of the fluid such that fluid flows through both the first passage 112 and the second passage 114. When fluid flows in the second direction 124, the valve 120 is forced to close, as shown in FIG. 6, under the action of the fluid such that fluid can only flow through the second passage 114 as the valve 120 closes the aperture 126 of the first passage 112 preventing fluid passage therethrough.

In examples of the invention, when the valve 120 is in the open condition fluid can be conveyed via the first passage 112, and when the valve 120 is in the closed condition fluid cannot be conveyed via the first passage 112. The tube may comprise a plurality of passages, one or more of which is prevented from conveying fluid when the valve 120 is in the closed condition, whilst one or more of the passages may allow the passage of fluid when the valve 120 is in the closed condition.

In the example of FIG. 5 and FIG. 6, when the valve 120 is in the open condition, fluid can be conveyed via both the first passage 112 and the second passage 114, and when the valve 120 is in the closed condition, fluid cannot be conveyed via the first passage 112, but can only be conveyed via the second passage 114. Given a fluid transfer system where the same volume of fluid must pass through the tube 110 in the first direction 122 and the second direction 124, for example in a sleep apnoea screening device where a user breathes in and out through a mask connected to the tube 110, the closing of the valve 120 to prevent fluid passing through the first passage 112 when fluid flow through the tube 110 is in the second direction 124 means that all of the fluid passes through the second passage 114. In such a scenario, a different fluid pressure is present at the pressure sensor connection point 116 during fluid flow in the first direction 122 and fluid flow in the second direction 124, thus providing an indicator of the direction of flow of the fluid, making the direction of fluid flow easier to detect using only a single sensor.

FIG. 7 shows a cross section of the tube 10 through section X3-X4 of FIG. 5. In this example the tube 110 is circular, or annular providing a circular inner channel, in cross section. However, rather than having concentric passages 12, 14 for the conveyance of a fluid as illustrated in FIG. 1, the first passage 112 may be a first segment 128 of the tube 110 in cross section forming a first part of the tube 110, the second passage 114 may be a second segment 130 of the tube 110 in cross section forming a second part of the tube 110.

FIG. 8 shows a cross section of the tube 110 through section Y3-Y4 of FIG. 6. In this example, where the valve 120 is in the closed position, the valve 120 is a segment in cross section. The valve 120, which may be formed of a flexible material to move from the open condition shown in FIG. 5 to the closed condition in FIG. 6, has a cross section encompassing substantially the same cross section as the first passage 112. Thereby, when the valve 120 is in the closed condition the first passage 112 is entirely closed off from the fluid path and so fluid conveyed in the second direction 124 can only pass through the second passage 114.

Therefore, between passage 112 and passage 114 is a chord of the tube 110. In the embodiment shown in FIG. 5 the chord is a diameter of the tube 110, however in other embodiments the chord may not be the diameter of the tube 110. In the embodiment shown in FIG. 7, the first segment 128 is a semi-circle forming a first half of the tube 110 in cross section and the second segment 130 is a semi-circle forming a second half of the tube 110 in cross section. In FIG. 8, it is illustrated that the valve 120 is a semi-circle, substantially of the same dimensions as the first segment 128.

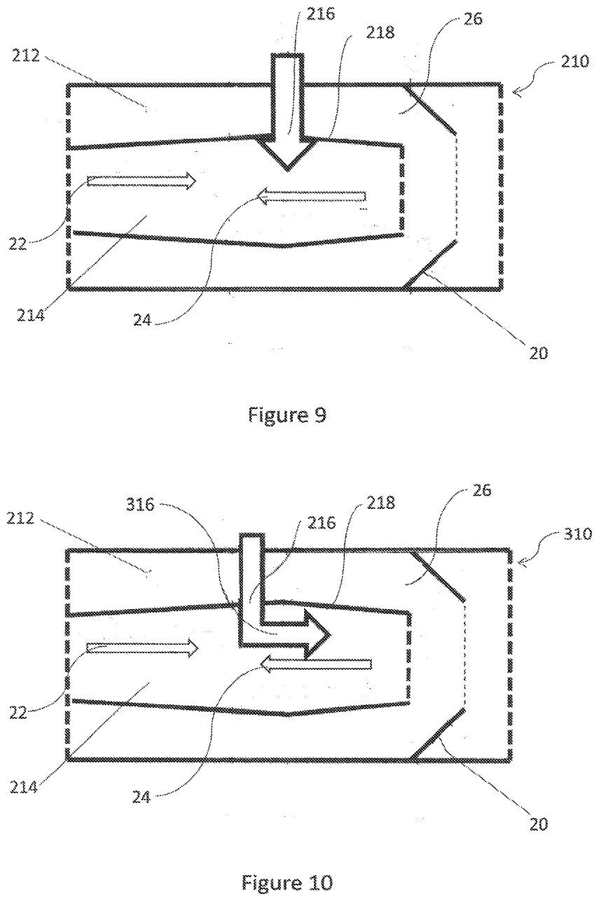

FIG. 9 illustrates a modification to the previously described embodiments, whereby the second passage 14, 114 may comprise an expansion chamber. Although shown in FIG. 9 relative to the embodiment of FIG. 1, it will be understood that the feature of an expansion chamber can also be applied to the embodiment of FIG. 5.

FIG. 9 modifies the example of FIG. 1 by modifying the wall 18 of the second passage 14, to be an expanded wall 218, with a pressure sensor connection point 216, providing a widened portion of the second passage 14 and a narrowed section of the first passage 12 to produce a tube 210 with an expansion chamber 214 and a flow constriction section 212. The effect of the expansion chamber 214 is to modify the fluid pressure observed at the pressure sensor connection point 216 during fluid flow in the first direction 22 and fluid flow in the second direction 24.

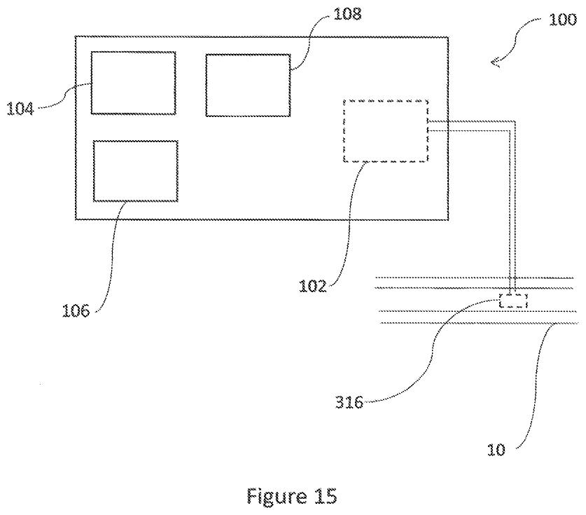

FIG. 10 illustrates another modification that can be provided in the tube 310. Although shown relative to the embodiment of FIG. 9, it will be understood that the modification illustrated in FIG. 10 may be applied to any other embodiment of this disclosure. Whereas FIGS. 1 to 9 illustrate a pressure sensor connection point 16, 116, 216 in the form of a tap or tapping point at which point a further tube or pipe may be connected to facilitate the connection to a sensor 102, as illustrated in FIG. 15, FIG. 10 illustrates a pressure sensor 316 positioned within the flow of fluid in the second passage 14, 114, or expansion chamber 214. That is tube 310 may comprise a pressure sensor 316 attached to the pressure sensor connection point 216 for measurement of pressure of the fluid flow through the second passage 14, 114, or expansion chamber 214 of the tube 310. The pressure sensor 316 may be any device that translates a magnitude of a physical pressure that is being exerted on a sensing element of the pressure sensor 316 into an output signal that can be used to establish a quantitative value for the pressure in the passage 14, 114, or expansion chamber 214.

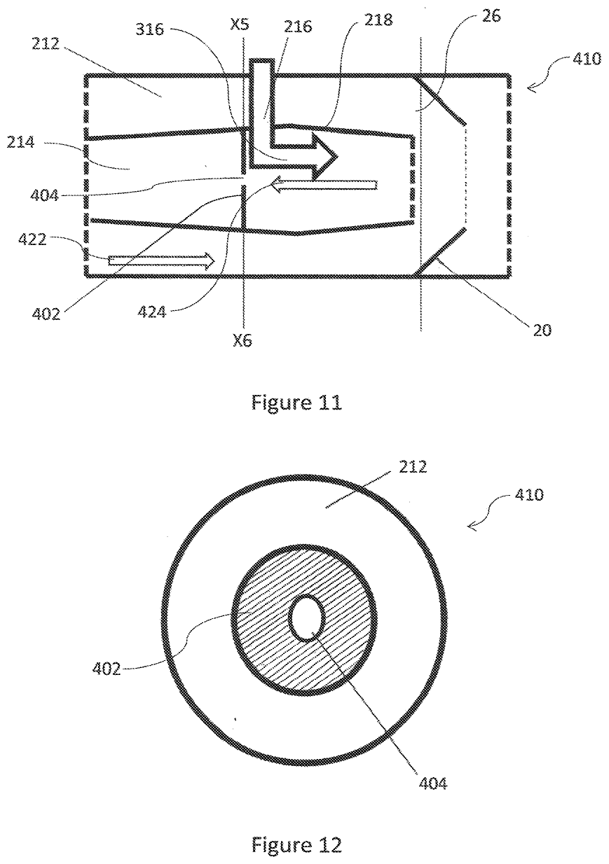

FIG. 11 illustrates another modification that can be provided in the tube 410. Although shown relative to the embodiment of FIG. 10, it will be understood that the modification illustrated in FIG. 11 may be applied to any other embodiment of this disclosure.

In FIG. 11, the second passage 214 is provided with a restrictor plate 402 upstream of the pressure sensor 316 in the fluid flow in a first direction 422. Fluid flow in a second direction 424 opposite to the first direction 422 can present fluid to the pressure sensor 316, however the restrictor plate 402 restricts passage of fluid being conveyed further downstream of the restrictor plate 402 in the second direction 424 of fluid flow. Fluid flow may only pass through the restrictor plate 402 via a restrictor aperture 404 in the restrictor plate 402. The second passage 214 may therefore comprise a restrictor plate 402 such that the pressure sensor connection point 216 is between the restrictor plate 402 and the valve 20.

FIG. 12 shows a cross section of the tube 410 through section X5-X6 of FIG. 11. In this example the tube 410 is circular, or annular providing a circular inner channel, in cross section. In this example the first passage 212 is annular in cross section, that is, the first passage 212 is an annular section of the tube 410. Inboard of the first passage 212 is the restrictor plate 402, which restricts the second passage 214 of the tube 410. The restrictor plate 402 may be annular or substantially annular in cross section, with a restrictor aperture 404 defining a central portion of the restrictor plate 402, the restrictor aperture 404 being circular or substantially circular in cross section. The restrictor aperture 404 may be oval, or another shape, in cross section. The combination of the restrictor plate 402 and restrictor aperture 404 defines a circular section within the tube 410. Fluid flow in a second direction 424 opposite to the first direction 422 can present fluid to the pressure sensor 316, however the restrictor plate 402 with restrictor aperture 404 restricts passage of fluid being conveyed further downstream of the restrictor plate 402 in the second direction 424 of fluid flow, thereby modifying the pressure in the second passage 214.

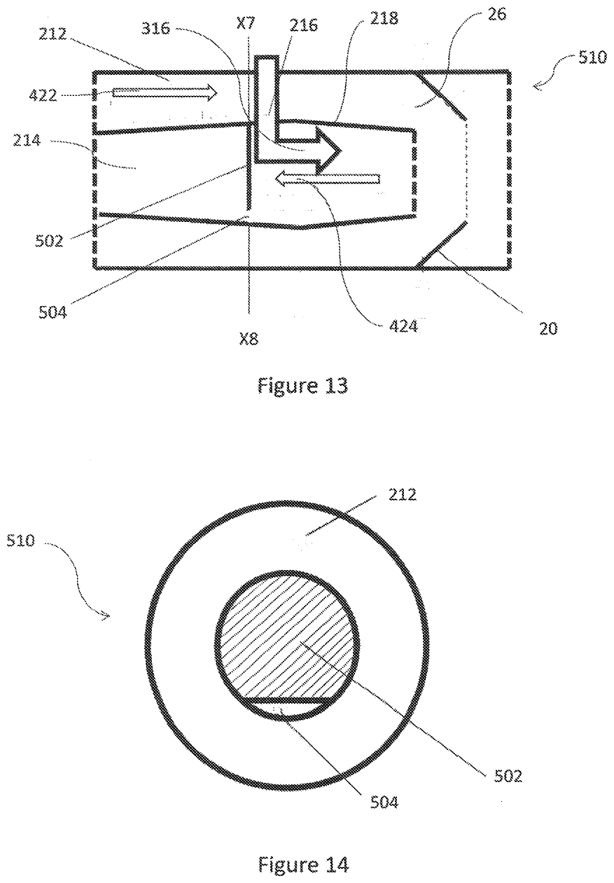

FIG. 13 illustrates a modification of the embodiment of FIG. 11 and FIG. 12, where the restrictor plate 502 is not circular or annular in cross section. The restrictor plate 502 may have a small restrictor aperture 504 therethrough, which, in embodiments where the fluid is a gas, may be a moisture drain hole 504. The drain hole 504 may be suitable for draining a liquid condensate from the tube 510. Fluid flow in a second direction 424 opposite to the first direction 422 can present fluid to the pressure sensor 316, however the restrictor plate 502 with drain hole 504 restricts passage of fluid being conveyed further downstream of the restrictor plate 502 in the second direction 424 of fluid flow, thereby modifying the pressure in the second passage 214.

FIG. 14 shows a cross section of the tube 510 through section X7-X8 of FIG. 13. In this example the tube 510 is circular, or annular providing a circular inner channel, in cross section. In this example the first passage 212 is annular in cross section, that is, the first passage 212 is an annular section of the tube 510. Inboard of the first passage 212 is the restrictor plate 502 with a drain hole 504. The restrictor plate 402 forms a major segment of the circle defined by the inner channel of the tube 510 and the drain hole 504 forms a minor segment of the circle defined by the inner channel of the tube 510. Although the division between the restrictor plate 502 and the drain hole 504 is illustrated as a chord, any other shape of drain hole 504 may be provided, though its positioning at the wall 218 of the second passage 214 allows for optimal fluid draining.

FIG. 15 illustrates a device 100 comprising a tube 10, 110, 210, 310, 410, 510 according to any preceding embodiment illustrated as a tube 10, in the form shown in FIG. 1, without detail of valves 20, expansion chambers 214, restrictor plates 402, 502, restrictor apertures 404 and drain holes 504 for simplicity. The device may be an obstructive sleep apnoea device 100 comprising a tube 10, 110, 210, 310, 410, 510. The device 100 further comprises a processor 108 for receiving signals from a sensor, a memory 104, and a power source 106. In embodiments where a sensor 316 is not provided in the fluid flow within the tube 10, 110, 210 a sensor 102 may be provided at the device 100 outside of the tube 10, 110, 210. In embodiments where the sensor 316 is provided in the fluid flow within the tube 310, 410, 510, then an additional sensor 102 may not be required.

Whilst the above embodiments have been described in relation to a tube, they may equally apply to a pipe or any other fluid conveying apparatus. The tube, pipe, or other fluid conveying apparatus may be any shape in cross section, such as circular, square, rectangular, oval, or any other shape. The above embodiments may also describe a container to be used as part of a fluid conveying system, that is, the tube as described may define a container in the form of a multi chamber container, which is located as part of a fluid conveying system, and which may therefore be connected to tubes or pipes for further conveyance of fluid to and from the container.

Although embodiments of the present invention have been described in the preceding paragraphs with reference to various examples, it will be appreciated that various changes and modifications to the examples can be made without departing from the scope of the invention as claimed.

Features described in the preceding description may be used in combinations other than the combinations explicitly described.

Although functions have been described with reference to certain features, those functions may be performable by other features whether described or not.

Although features have been described with reference to certain embodiments, those features may also be present in other embodiments whether described or not.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A tube for conveying a fluid, the tube comprising:
a first passage;
a second passage comprising a pressure sensor connection point through a wall of the second passage; and
a valve configured to be in an open condition when a fluid flow through the tube in is a first direction and configured to be in a closed condition when the fluid flow through the tube is in a second direction, opposite to the first direction, wherein the pressure sensor connection point is upstream of the valve, when the fluid flow is in the first direction, and downstream of the valve, when the fluid flow is in the second direction;
wherein, when the valve is in the open condition fluid can be conveyed via the first passage, and when the valve is in the closed condition fluid cannot be conveyed via the first passage.

2. The tube according to claim 1, wherein, when the valve is in the open condition fluid can be conveyed via the first passage and the second passage, and when the valve is in the closed condition fluid can only be conveyed via the second passage.

3. The tube according to claim 1, comprising: a pressure sensor attached to the pressure sensor connection point for measurement of pressure of the fluid flow through the second passage of the tube.

4. The tube according to claim 3, wherein the pressure sensor has a pressure sensing element in the flow of fluid inside of the second passage.

5. The tube according to claim 3, wherein the pressure sensor connection point is a pressure sensor tap, the pressure sensor tap being connected to a pressure sensing element outside of the second passage.

6. The tube according to claim 1, wherein the tube is circular in cross section.

7. The tube according to claim 6, wherein the first passage is annular in cross section and has a first outer diameter, the second passage is circular in cross section and has a second outer diameter, the second passage being concentric with the first passage, the second outer diameter being less than the first outer diameter, and the valve is annular in cross section, having a valve outer diameter in the closed condition that is substantially equal to the first outer diameter and an valve inner diameter in the closed condition that is substantially equal to the second outer diameter.

8. The tube according to claim 6, wherein the first passage is a first segment of the tube in cross section forming a first part of the tube, the second passage is a second segment of the tube in cross section forming a second part of the tube, and the valve is a segment in cross section when in the closed condition, encompassing substantially the same cross section as the first passage.

9. The tube according to claim 8, wherein the first segment is a semi-circle forming a first half of the tube in cross section and the second segment is a semi-circle forming a second half of the tube in cross section.

10. The tube according to claim 1, wherein the second passage comprises an expansion chamber.

11. The tube according to claim 1, wherein the second passage comprises a restrictor plate such that the pressure sensor connection point is between the restrictor plate and the valve.

12. The tube according to claim 11, wherein the restrictor plate comprises a drain hole for draining a liquid condensate from the tube.

13. A device comprising the tube according to claim 3, and further comprising a processor for receiving signals from the pressure sensor, a memory and power source.
